(19) United States
(12) Patent Application Publication
(10) Pub. No.: US 2009/0118628 A1
(43) Pub. Date: May 7, 2009

(54) SYSTEM FOR MEASURING BLOOD PRESSURE FEATURING A BLOOD PRESSURE CUFF COMPRISING SIZE INFORMATION

(75) Inventors: Zhou ZHOU, San Diego, CA (US); Matthew J. BANET, Del Mar, CA (US)

Correspondence Address: WilmerHale/Triage Wireless, 60 State Street, Boston, MA, 02109, US

(73) Assignee: TRIAGE WIRELESS, INC., San Diego, CA (US)

(21) Appl. No.: 12/262,689

(22) Filed: Oct. 31, 2008

Related U.S. Application Data
(60) Provisional application No. 60/984,424, filed on Nov. 1, 2007.

(51) Int. Cl. A61B 5/022

(52) U.S. Cl. 600/499

(57) ABSTRACT

A system for measuring blood pressure is described that includes a blood pressure cuff with a sizing indicator. The sizing indicator presents size information indicating either the size of the blood pressure cuff or the size of a patient's arm within the blood pressure cuff. The system also includes a monitor featuring a sensing component that senses the size information from the sizing indicator. A pressure-monitoring system, which is coupled to the blood pressure cuff and may be in wireless communication with the monitor, measures a pressure signal from the patient's arm. The pressure-monitoring system is coupled to a processor that processes both the pressure signal and the size information to measure the patient's blood pressure.

This application claims the benefit of U.S. Provisional Application No. 60/984,424, filed Nov. 1, 2007, all of which is incorporated herein by reference.

### TECHNICAL FIELD

The present invention relates to medical devices for monitoring vital signs, e.g., arterial blood pressure.

### BACKGROUND OF THE INVENTION

Blood within a patient's body is characterized by a baseline pressure value, called the diastolic pressure. A heartbeat forces a time-dependent volume of blood through the artery, causing the baseline pressure to increase in a pulsatile manner to a value called the systolic pressure. The systolic pressure indicates a maximum pressure in a portion of the artery that contains a flowing volume of blood. Pulse pressure is the difference between systolic and diastolic pressure. Mean blood pressure represents a mathematical mean between systolic and diastolic pressure, and is approximately equal to diastolic pressure plus one third of the pulse pressure.

Both invasive and non-invasive devices can measure a patient's systolic and diastolic blood pressure. A non-invasive medical device called a sphygmomanometer measures a patient's blood pressure using an inflatable cuff (e.g. a plastic coated nylon material with an embedded air bladder) and a sensor (e.g., a stethoscope) according to a technique called auscultation. During auscultation, a medical professional rapidly inflates the cuff to a pressure that exceeds the patient's systolic blood pressure. The medical professional then slowly deflates the cuff, causing the pressure to gradually decrease, while listening for flowing blood with the stethoscope. Sounds called the ‘Korotkoff sounds’ indicate both systolic and diastolic blood pressure. Specifically, the pressure value at which blood first begins to flow past the deflating cuff, indicated by a first Korotkoff sound, is the systolic pressure. The stethoscope monitors this pressure by detecting strong, periodic acoustic ‘beats’ or ‘taps’ indicating that the systolic pressure barely exceeds the cuff pressure. The minimum pressure in the cuff that restricts blood flow, as detected by the stethoscope, is the diastolic pressure. The stethoscope monitors this pressure by detecting another Korotkoff sound, in this case a ‘leveling off’ or disappearance in the acoustic magnitude of the periodic beats, indicating that the cuff no longer restricts blood flow.

Automated blood pressure monitors use a technique called oscillometry to measure blood pressure. Most monitors using oscillometry rapidly inflate the cuff, and then measure blood pressure while the cuff slowly deflates. During deflation, mechanical pulsations corresponding to the patient's heartbeats couple into the cuff as the pressure reduces from systolic to diastolic pressure. The pulsations modulate the pressure waveform so that it includes a series of time-dependent pulses, with the amplitude of the pulses typically varying with applied pressure. Processing the pressure waveform with well-known digital filtering techniques typically yields a train of pulses characterized by a Gaussian or similar distribution; the maximum of the amplitude distribution corresponds to mean arterial pressure. Diastolic and systolic pressures are determined from, respectively, the rising and falling sides of the Gaussian distribution. Typically diastolic pressure corresponds to an amplitude of 0.55 times the maximum amplitude, while systolic pressure corresponds to an amplitude of 0.72 times the maximum amplitude.

Both auscultation and oscillometric blood pressure measurements depend in part on the size of the blood pressure cuff relative to the patient's arm circumference. A cuff that is too large or too small influences the blood pressure measurement and can result in inaccuracies. Typical adult blood pressure cuffs come in at least 4 standard sizes: adult small (arm circumference less than 27 cm), adult (27-34 cm), adult large (35-44 cm), and adult thigh cuff (45-52 cm).

Auscultation and oscillometric blood pressure measurements are well-known in the art, and are described by a number of issued U.S. Pat. Nos. 4,112,929; 4,592,365; and 4,627,440.

### SUMMARY OF THE INVENTION

The described embodiments provide a system for measuring blood pressure that accounts for either the type of cuff, typically made of a plastic coated nylon material with an inflatable air bladder, used during the measurement (e.g. adult small, adult, adult large, adult thigh cuff) or the specific circumference of the patient's arm, and then uses this information in a subsequent blood pressure measurement. In this way, the system optimizes the measurement or corrects for a measurement bias that depends on either the cuff size of the patient's arm circumference.

In one aspect, for example, the system features a subsystem for measuring blood pressure that includes a blood pressure cuff with a sizing indicator. The sizing indicator describes size information indicating either the size of the blood pressure cuff or the size of a patient's arm within the blood pressure cuff. The system also includes a monitor featuring a sensing component that senses the size information from the sizing indicator. A pressure-monitoring system, which is coupled to the blood pressure cuff and may be in wireless communication with the monitor, measures a pressure signal from the patient's arm. The pressure-monitoring system is coupled to a processor that processes both the pressure signal and the size information to measure the patient's blood pressure.

In embodiments, the sizing indicator on the blood pressure cuff is a barcode label, and the sensing component on the monitor is a barcode scanner. The pressure-monitoring system typically includes a motor-controlled pump, and the processor operates an algorithm that, after processing the size information, controls the rate at which the pump inflates the blood pressure cuff. The algorithm can further adjust this rate with a closed-loop feedback system that detects the rate at which the cuff is being inflated, and then further adjusts the inflation rate. Typically both the monitor and the pressure-monitoring system each include a wireless transceiver. In this embodiment, during a measurement, the wireless transceiver in the pressure-monitoring system receives a signal indicating a size of the blood pressure cuff sensed by the sensing component on the monitor.

In other embodiments the blood pressure cuff includes a flexible strap featuring a size indicator configured to indicate a circumference of the patient's arm once the blood pressure cuff is wrapped around the patient's arm. Typically, in this embodiment, the blood pressure cuff includes a plurality of size indicators, each one indicating a different arm circumference. A marker indicates a specific size indicating an arm circumference once the blood pressure cuff is wrapped around the patient's arm. In this case the processor operates an algorithm that processes the signal indicating arm circumference to control the rate at which the pump inflates the blood pressure cuff. Alternatively, the signal indicating arm circumference is processed to generate a blood pressure offset value that is used to adjust a blood pressure value.

As an alternative to a barcode label, the blood pressure cuff can include a sizing indicator featuring an alphanumeric code (e.g. an RFID) that encodes size information indicating the size of the blood pressure cuff. In this case the monitor features a matched sensing component (e.g. an RFID reader) that wirelessly senses the alphanumeric code. In still other embodiments the monitor features a touchpanel display that renders a graphical user interface wherein the user can manually enter sizing information from the blood pressure cuff. For example, the user interface can include a pull-down menu wherein the user can select specific size information from a plurality of fields, each indicating different cuff sizes or arm circumferences.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 shows a schematic drawing of the system described herein featuring a hand-held monitor with a barcode scanner and a cuff labeled with a barcode indicating its size.

FIG. 2A shows a graph of time-dependent pressure waveforms measured during an inflation-based blood pressure measurement using three different inflation rates: a rate that is too fast (A); a rate that is correct (B); and a rate that is too slow (C).

FIG. 2B shows a graph of the time-dependent pressure waveforms of FIG. 2A, processed with a signal processing algorithm to yield pulsation amplitude as a function of applied pressure.

FIG. 3 shows an adjustable blood pressure cuff with a barcode label that, when used with the system described herein, yields a patient's arm circumference for use in a blood pressure measurement.

FIGS. 4A and 4B show, respectively, front and back sides of the adjustable blood pressure cuff of FIG. 3.

FIG. 5 shows a hand-held monitor of FIG. 1 that scans the barcode on the adjustable blood pressure cuff of FIG. 3.

FIG. 6 shows a graph of blood pressure as a function of arm circumference.

FIG. 7 shows a body-worn sensor for inflating and deflating the adjustable cuff of FIG. 3.

### DETAILED DESCRIPTION

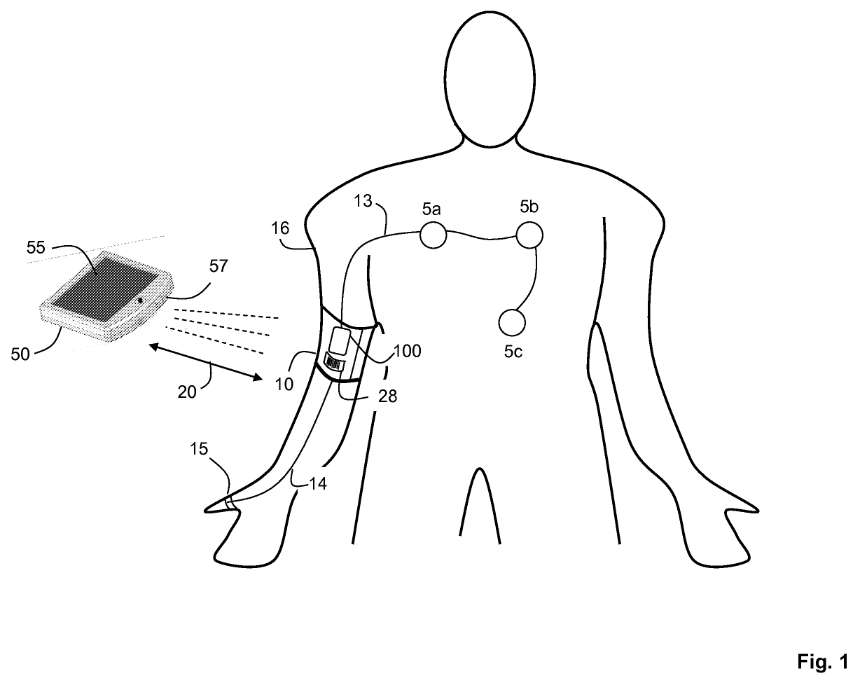

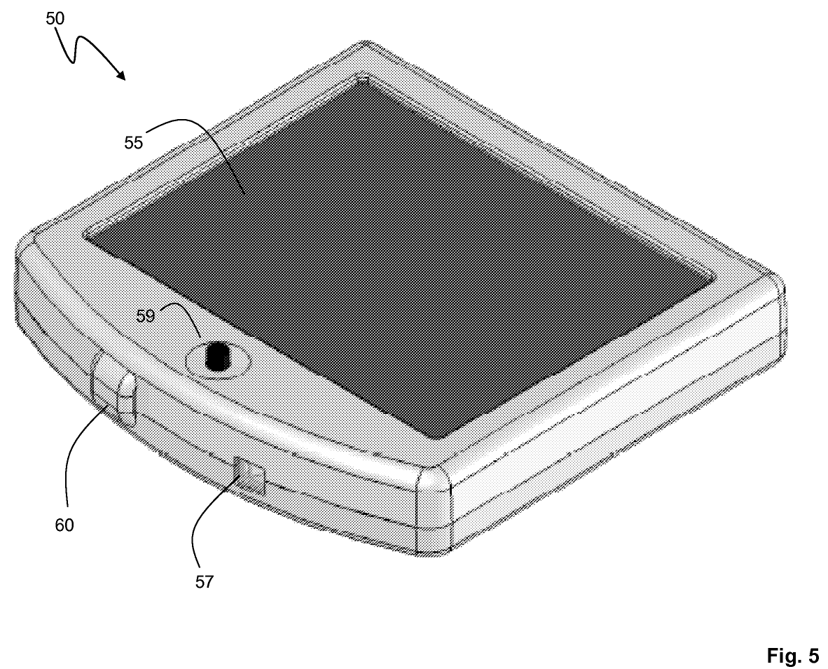

FIGS. 1 and 5 shows a schematic drawing of the system described herein featuring a hand-held monitor 50 that works in concert with a specialized cuff 10 and body-worn sensor 100 to measure blood pressure from a patient's arm 16. Typically the system is used to make inflation-based oscillometric measurements, as is described in more detail below. The specialized cuff 10 includes a barcode label 28 indicating its size (e.g. adult small, adult, adult large, adult thigh cuff). During a measurement, it is inflated by a mechanical pump, solenoid value, and control system within the body-worn sensor 100. The monitor 50 features an internal Bluetooth receiver, a barcode scanner 57, and a touchpanel display 55 that renders an icon-driven graphical user interface. Prior to making a blood pressure measurement, a medical professional controls the monitor 50 and barcode scanner 57 through the touchpanel display 55 to scan the barcode label 28 adhered to the cuff 10. This yields the cuff size, which can then be processed during an inflation-based blood pressure measurement to control the inflation rate, thereby increasing the accuracy of the blood pressure measurement.

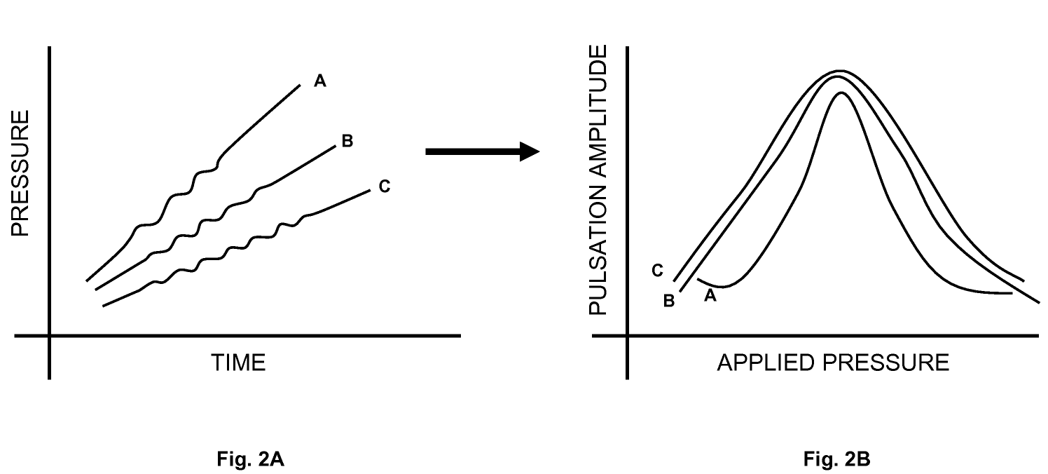

Inflation-based oscillometric blood pressure measurements can be preferable from the patient's point of view, as they are typically faster and more comfortable than conventional deflation-based oscillometric measurements. Such measurements typically use a mechanical pump to rapidly inflate a cuff worn of a patient's arm, and a solenoid value to then slowly deflate the cuff while a pressure sensor measures a pressure waveform. In an inflation-based oscillometric measurement, like the one described herein, the mechanical pump slowly inflates the cuff, during which the control system within the body-worn sensor measures and processes the pressure waveform. Once the measurement is complete, the control system commands the solenoid valve to open to rapidly exhaust the cuff. Ideally the body-worn sensor 100 described herein inflates the cuff 10 at a linear rate between 4-7 mmHg/second. Smaller cuffs (characterized by the ‘small adult’ size) tend to inflate relatively fast, while larger cuffs (characterized by the ‘adult large’ or ‘adult thigh cuff’ sizes) tend to inflate relatively slow. Both these conditions, as described below with reference to FIGS. 2A and 2B, can decrease the accuracy of the blood pressure measurement. To combat this problem, the current system adjusts the inflation rate of the cuff 100 based on its size, determined using the barcode label 28 and barcode scanner 57. Once the size is determined, the control system within the body-worn sensor 100 modulates the voltage applied to the pump (typically by adjusting the duty cycle of the voltage using pulse wave modulation) to carefully control the inflation rate. After an initial rate is set, the control system can slightly adjust it during the course of the oscillometric measurement using a closed-loop pressure-monitoring system. In this way the inflation rate can be kept linear, which is ideal for optimizing the accuracy of the blood pressure measurement.

Referring to FIGS. 1 and 5, the barcode scanner 57 is ideal for reading the cuff's size from the barcode label 28, as it does not require the medical professional to input any information into the monitor. Other approaches can be also used. For example, the cuff's size can be manually input into the system through the monitor's touchpanel 55, or can be encoded on an RFID chip embedded in the cuff, and then read with an RFID reader in the monitor. Both the monitor 50 and the body-worn sensor 100 feature embedded Bluetooth transceivers, and communicate wirelessly as indicated by the arrow 20. The monitor 50 can additionally include an external antenna 60 to increase the range of the Bluetooth communication. In this way, the cuff's size can be determined by the monitor 100 as described above, and then sent wirelessly to the body-worn sensor 100 to control the inflation rate. The monitor 50 is powered on and off with a simple push-button switch 59.

In addition to making occasional inflation-based oscillometric measurements, the above-described system can continuously measure blood pressure from the patient using a technique based on a ‘pulse transit time’ determined from three ECG electrodes 5a-c attached to the patient's chest, and an optical sensor 15 attached to the patient's thumb. Pulse transit time is inversely related to blood pressure, and is determined from the time difference separating a QRS complex in the electrical waveform, and the foot of a pulse in the optical waveform. In the current system, these waveforms are determined from ECG electrodes 5a-c and an optical sensor 15 that connect to the body-worn sensor 100 through cables 13 and 14, respectively. A preferred technique and body-worn sensor for continuously measurement blood pressure are described in the co-pending patent application entitled: VITAL SIGN MONITOR MEASURING BLOOD PRESSURE USING OPTICAL, ELECTRICAL, AND PRESSURE WAVEFORMS (U.S. Ser. No. 12/138,194; filed Jun. 12, 2008), the contents of which are incorporated herein by reference. The body-worn sensor featured in this patent application is described briefly below.

FIGS. 2A and 2B show graphs that illustrate the importance of carefully controlling a cuff's inflation rate during an inflation-based oscillometric measurement. Specifically, FIG. 2A shows three time-dependent pressure waveforms (A, B, and C) that are characteristic of those measured from a patient's arm with the above-described system. Each waveform features a series of pulses superimposed on a time-dependent pressure that increases in a mostly linear fashion. As described above, the pulses represent mechanical pulsations corresponding to the patient's heartbeats that couple into the cuff as the pressure increases from diastolic to systolic pressure. To determine blood pressure, a microprocessor in the body-worn sensor's control system processes the time-dependent pressure waveform with 2-stage digital filtering process. The first stage has a pass band typically between 0.5-7.0 Hz, and yields a train of pulses, each corresponding to a unique heartbeat, characterized by an envelope having Gaussian-type distribution. The second stage has a pass band typically between 0.1 and 0.4 Hz and, as shown in FIG. 2B, yields only the smoothed envelope. If the pump increases the pressure too quickly in the cuff, as shown by pressure waveform A and is typical of adult small cuffs, not enough heartbeat-induced pulsations are included in the waveform shown in FIG. 2A. The resulting waveform, following processing with the 2-stage digital filtering process, is shown in FIG. 2B. It is artificially narrow because of the lack of pulsations; this typically results in a systolic blood pressure that is too low, and diastolic pressure that is too high. If the pump increases the pressure in the cuff too slowly, as indicated by pressure waveform C, the measurement can be drawn out in time, which can be uncomfortable to the patient. Additionally, this can cause too many oscillations in the pressure waveform, which can artificially broaden the Gaussian-type waveform shown in FIG. 2B. This can, respectively, erroneously increase systolic pressure and decrease diastolic pressure, although the errors are typically less than those incurred when the inflation is too fast. Pressure waveform B is ideal, and, as described above, is characterized by a pressure increase of between 4-7 mmHg/second.

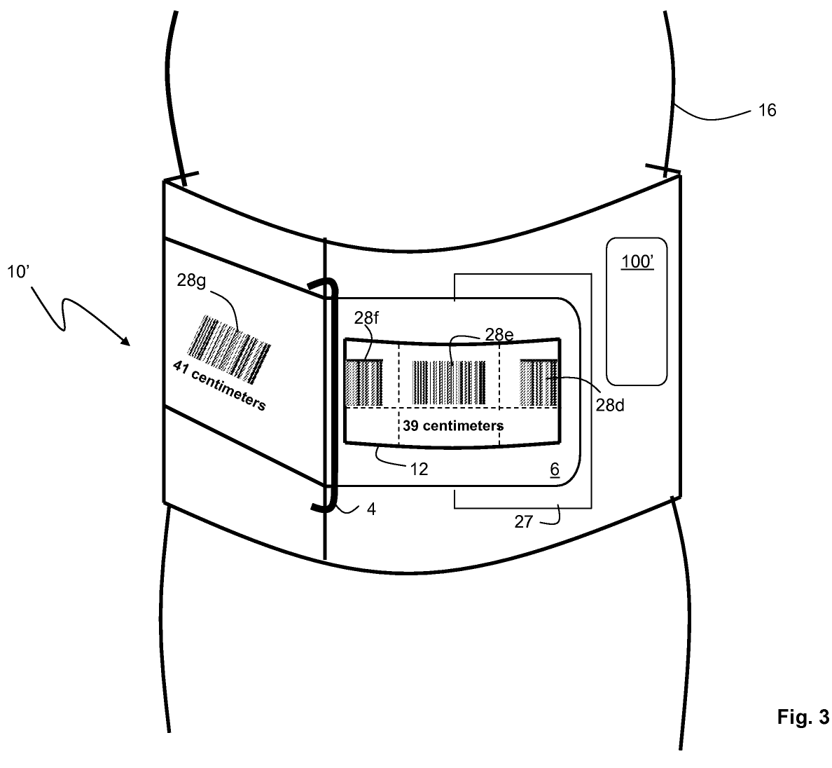

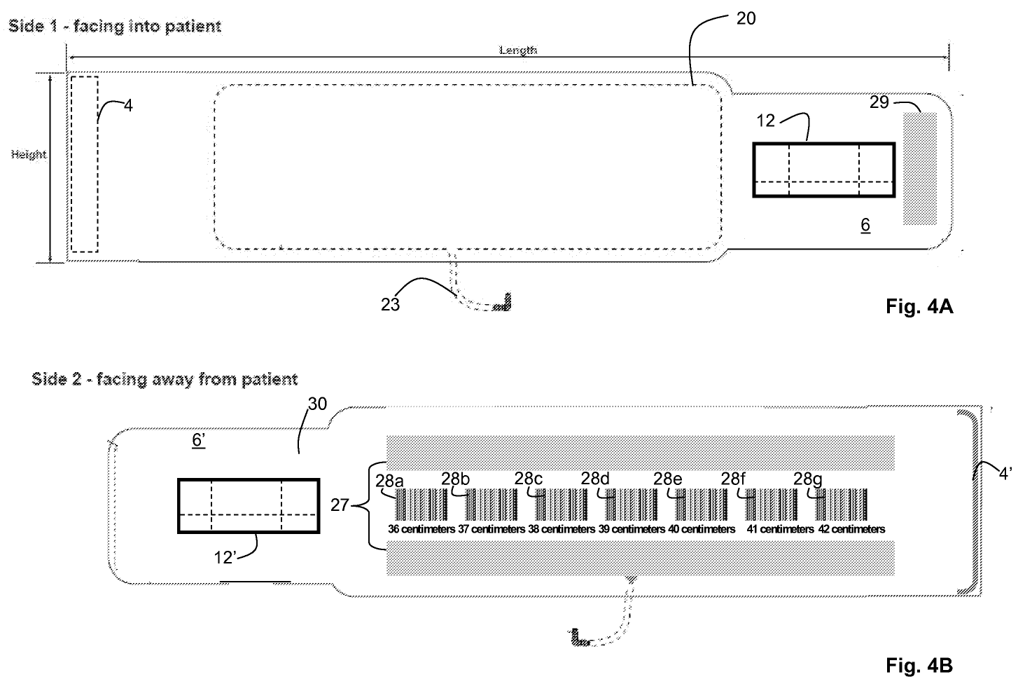

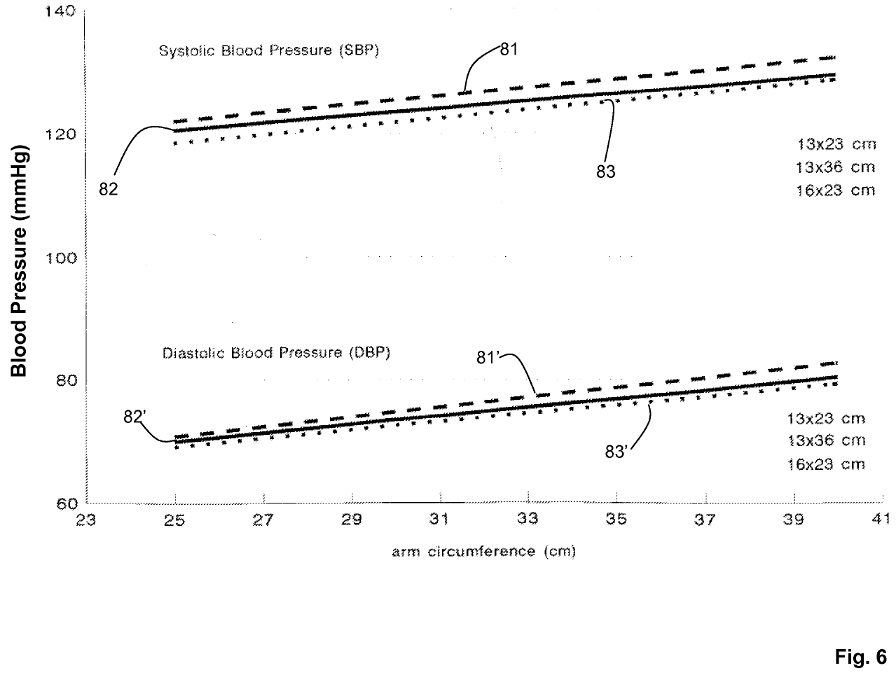

Another embodiment of the above-described system is shown in FIGS. 3, 4A, and 4B. These figures show schematic drawings of an adjustable blood pressure cuff 10′ that connects to a motor-controlled pump through a pneumatic cable 23 and includes a series of printed barcodes 28a-g indicating the patient's arm circumference. Similar to the cuff size, this parameter can then be used in a calculation to improve accuracy of the blood pressure measurement. Typically the cuff 10′ is a nylon material coated in a plastic composite (e.g. Polyvinyl chloride or commonly known as ‘PVC’) and contains an inflatable air bladder 20 that inflates and deflates during a measurement. During operation, the medical professional wraps the adjustable blood pressure cuff 10′ tightly around the patient's arm 16 by looping a tapered window flap 6, 6′ through a D-ring 4, 4′, and folding it back so that a Velco® patch 29 proximal to the D-ring 4, 4′ adheres to a matched Velco® strip 27. The window flap 6, 6′ contains a clear, flexible window 12, 12′ that aligns with each barcode 28a-g according to the patient's arm circumference. A numerical value representing each circumference is encoded within the appropriate barcode 28a-g. As described above, prior to a measurement, a monitor similar to that described with reference to FIG. 1 scans the barcode value underneath the clear, flexible window 12, 12′ using the barcode scanner. This incorporates the patient's arm circumference into firmware running on the monitor, which then uses it during an inflation-based oscillometric measurement to add an offset to the calculated systolic and diastolic blood pressure values. FIG. 6 shows a graph from which the exact offset values can be determined. Once the barcode 28a-g is scanned, the monitor sends a wireless signal to the control system within the body-worn sensor, which initiates the blood pressure measurement. Pressure values measured by the body-worn sensor are wirelessly sent back to the monitor, which processes them and the patient's arm circumference to determine blood pressure. Once determined, this value is then rendered on the monitor's touchpanel display.

Alternatively, the monitor can scan the cuff's barcode 28a-g, and then transmit this value through Bluetooth to the body-worn sensor. A microprocessor in the body-worn sensor then uses this value and pressure values measured by the pressure sensor to calculate an accurate blood pressure value.

A monitor like that described above has been described previously by Applicants in: BLOOD PRESSURE MONITOR (U.S. Ser. No. 11/530,076; filed Sep. 8, 2006) and MONITOR FOR MEASURING VITAL SIGNS AND RENDERING VIDEO IMAGES (U.S. Ser. No. 11/682,177; filed Mar. 5, 2007), the contents of which are incorporated herein by reference. In some applications it may be required to ‘pair’ the monitor with the body-worn sensor. This ensures an exclusive, one-to-one relationship between these two components, thus prohibiting the monitor from receiving signals from an extraneous body-worn sensor. Pairing is typically done with the monitor's barcode scanner. During operation, a user holds the monitor in one hand, and points the barcode scanner at a printed barcode on the body-worn sensor. This includes information (e.g. a MAC address an PIN) describing its internal Bluetooth transceiver. Once the information is received, software running on microprocessors within both the monitor and body-worn sensor analyzes it to complete the pairing. This methodology forces the user to bring the monitor into close proximity to the body-worn sensor, thereby reducing the chance that vital sign information from another body sensor is erroneously received and displayed.

FIG. 6 shows a graph of systolic blood pressure (SBP) 81, 82, 83 and diastolic blood pressure (DBP) 81′, 82′, 83′ as a function of arm circumference measured from three different cuff sizes. These data were published in the following article, the contents of which are incorporated herein by reference: Bakx, C., Oelemans, G., van den Hoogen, H. et al., ‘The influence of cuff size on blood pressure measurement’, J of Hypertension. (1997) 11, 439-445. As shown in the graph, blood pressure values vary with the patient's arm circumference and the size of the measuring cuff. By including a calibration curve representing the data in FIG. 6 in firmware running on the monitor or body-worn sensor, the patient's arm circumference and the cuff size can be corrected for during a blood pressure measurement. Typically the patient's arm circumference is entered when the monitor's barcode scanner scans the cuff's barcode. This value is then used in the subsequent blood pressure calculation. When used with the blood pressure cuff shown in FIGS. 3, 4A, and 4B, this has the advantage that only a single cuff may be required for a wide range of arm circumferences. Typically the ideal ratio of the width of the cuff's bladder to the circumference of the patient's arm is about 0.40, as described in the thesis entitled ‘Transducer for Indirect Measurement of Blood Pressure in Small Human Subjects and Animals’, Roeder, Rebecca Ann, Purdue University. (2003). With the cuff described in FIGS. 3, 4A, and 4B, the exact ratio can be measured accurately for every patient; deviations from the ideal ratio of 0.40 can be corrected for each patient according to a pre-determined look-up table determined from the data shown in FIG. 6 to increase the accuracy of the measured blood pressure.

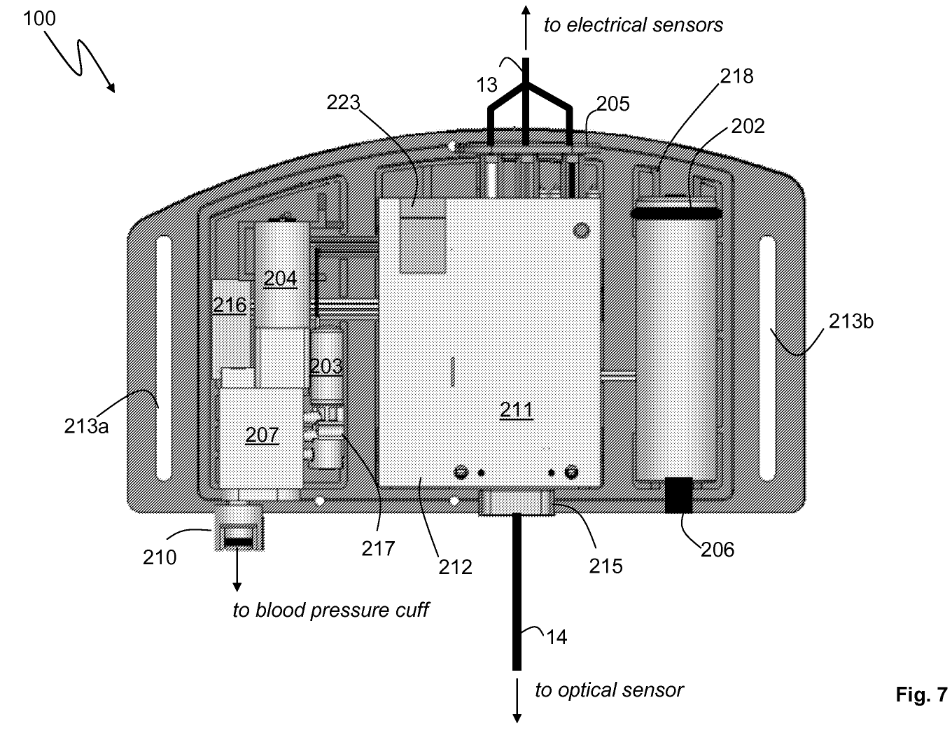

FIG. 7 shows a top view of the body-worn sensor 100 used to conduct the above-described measurements. The body-worn sensor 100 features a single circuit board 212 including connectors 205, 215 that connect through separate cables 13, 14 to, respectively, electrodes worn on the patient's body and optical sensor worn on the patient's hand. During a measurement of pulse transit time, these sensors measure electrical and optical signals that pass through connectors 205, 215 to discrete circuit components 211 on the bottom side of the circuit board 212. The discrete components 211 include: i) analog circuitry for amplifying and filtering the time-dependent optical and electrical waveforms; ii) an analog-to-digital converter for converting the time-dependent analog signals into digital waveforms; and a iii) microprocessor for processing the digital waveforms to determine blood pressure according to the above-described technique, along with other vital signs. The body-worn sensor 100 attaches to an arm-worn cuff using Velcro® through two D-ring loops 213a, 213b. The cuff secures the body-worn sensor 100 to the patient's arm.

To measure the pressure waveform during an inflation-based oscillometric measurement, the circuit board 212 additionally includes a small mechanical pump 204 for inflating the bladder within the cuff, and a solenoid valve 203 for controlling the bladder's inflation and deflation rates. The pump 204 and solenoid valve 203 connect through a manifold 207 to a connector 210 that attaches through a tube (not shown in the figure) to the bladder in the cuff, and additionally to a digital pressure sensor 216 that senses the pressure in the bladder. The solenoid valve 203 couples through the manifold 207 to a small ‘bleeder’ valve 217 featuring valve that controls air to rapidly release pressure. Typically the solenoid valve 203 is closed as the pump 204 inflates the bladder. For measurements conducted during inflation, pulsations caused by the patient's heartbeats couple into the bladder as it inflates, and are mapped onto the pressure waveform. The digital pressure sensor 216 generates an analog pressure waveform, which is then digitized with the analog-to-digital converter described above. The microprocessor processes the digitized pressure, optical, and electrical waveforms to determine systolic, mean arterial and diastolic blood pressures. Once these measurements are complete, the microprocessor immediately opens the solenoid valve 203, causing the bladder to rapidly deflate.

A rechargeable lithium-ion battery 202 mounts directly on the body-worn sensor's flexible plastic backing 218 to power all the above-mentioned circuit components. Alternately, the sensor's flexible plastic backing 218 additionally includes a plug 206 which accepts power from a wall-mounted AC adaptor. The AC adaptor is used, for example, when measurements are made over an extended period of time. A Bluetooth transmitter 223 is mounted directly on the circuit board 212 and, following a measurement, wirelessly transmits information to an external monitor. A rugged plastic housing (not shown in the figure) covers the circuit board 212 and all its components.

In addition to those methods described above, a number of additional methods can be used to calculate blood pressure. These are described in the following co-pending patent applications, the contents of which are incorporated herein by reference: 1) CUFFLESS BLOOD-PRESSURE MONITOR AND ACCOMPANYING WIRELESS, INTERNET-BASED SYSTEM (U.S. Ser. No. 10/709,015; filed Apr. 7, 2004); 2) CUFFLESS SYSTEM FOR MEASURING BLOOD PRESSURE (U.S. Ser. No. 10/709,014; filed Apr. 7, 2004); 3) CUFFLESS BLOOD PRESSURE MONITOR AND ACCOMPANYING WEB SERVICES INTERFACE (U.S. Ser. No. 10/810,237; filed Mar. 26, 2004); 4) VITAL SIGN MONITOR FOR ATHLETIC APPLICATIONS (U.S.S.N; filed Sep. 13, 2004); 5) CUFFLESS BLOOD PRESSURE MONITOR AND ACCOMPANYING WIRELESS MOBILE DEVICE (U.S. Ser. No. 10/967,511; filed Oct. 18, 2004); 6) BLOOD PRESSURE MONITORING DEVICE FEATURING A CALIBRATION-BASED ANALYSIS (U.S. Ser. No. 10/967,610; filed Oct. 18, 2004); 7) PERSONAL COMPUTER-BASED VITAL SIGN MONITOR (U.S. Ser. No. 10/906,342; filed Feb. 15, 2005); 8) PATCH SENSOR FOR MEASURING BLOOD PRESSURE WITHOUT A CUFF (U.S. Ser. No. 10/906,315; filed Feb. 14, 2005); 9) PATCH SENSOR FOR MEASURING VITAL SIGNS (U.S. Ser. No. 11/160,957; filed Jul. 18, 2005); 10) WIRELESS, INTERNET-BASED SYSTEM FOR MEASURING VITAL SIGNS FROM A PLURALITY OF PATIENTS IN A HOSPITAL OR MEDICAL CLINIC (U.S. Ser. No. 11/162,719; filed Sep. 9, 2005); 11) HAND-HELD MONITOR FOR MEASURING VITAL SIGNS (U.S. Ser. No. 11/162,742; filed Sep. 21, 2005); 12) CHEST STRAP FOR MEASURING VITAL SIGNS (U.S. Ser. No. 11/306,243; filed Dec. 20, 2005); 13) SYSTEM FOR MEASURING VITAL SIGNS USING AN OPTICAL MODULE FEATURING A GREEN LIGHT SOURCE (U.S. Ser. No. 11/307,375; filed Feb. 3, 2006); 14) BILATERAL DEVICE, SYSTEM AND METHOD FOR MONITORING VITAL SIGNS (U.S. Ser. No. 11/420,281; filed May 25, 2006); 15) SYSTEM FOR MEASURING VITAL SIGNS USING BILATERAL PULSE TRANSIT TIME (U.S. Ser. No. 11/420,652; filed May 26, 2006); 16) BLOOD PRESSURE MONITOR (U.S. Ser. No. 11/530,076; filed Sep. 8, 2006); 17) TWO-PART PATCH SENSOR FOR MONITORING VITAL SIGNS (U.S. Ser. No. 11/558,538; filed Nov. 10, 2006); 18) MONITOR FOR MEASURING VITAL SIGNS AND RENDERING VIDEO IMAGES (U.S. Ser. No. 11/682,177; filed Mar. 5, 2007); 19) DEVICE AND METHOD FOR DETERMINING BLOOD PRESSURE USING ‘HYBRID’ PULSE TRANSIT TIME MEASUREMENT (U.S. Ser. No. 60/943,464; filed Jun. 12, 2007); 20) VITAL SIGN MONITOR MEASURING BLOOD PRESSURE USING OPTICAL, ELECTRICAL, AND PRESSURE WAVEFORMS (U.S. Ser. No. 12/138,194; filed Jun. 12, 2008); and, 21) VITAL SIGN MONITOR FOR CUFFLESSLY MEASURING BLOOD PRESSURE CORRECTED FOR VASCULAR INDEX (U.S. Ser. No. 12/138,199; filed Jun. 12, 2008).

Functionality described herein can be implemented by code executing on a processor. The code may be embodied in firmware or stored on and read from a digital storage medium, such as RAM, ROM, a CD, etc.

Still other embodiments are within the scope of the following claims.

What is claimed is:

1. A system for measuring blood pressure of a patient, said system comprising:
a blood pressure cuff including a sizing indicator that presents size information about at least one of a size of the blood pressure cuff and a size of a patient's arm about which the blood pressure cuff is placed when in use;
a monitor component including an interface through which the size information from the sizing indicator is received;
an inflation system that inflates the blood pressure cuff, said inflation system also including a pressure sensor for generating a pressure signal which is a measure of the pressure applied the patient's arm by the inflation system; and
a processor programmed to use the pressure signal from the inflation system and the size information from the blood pressure cuff to determine the patient's blood pressure.

2. The system of claim 1, wherein the monitor component is a sensor unit to be worn on the patient's body to monitor signals from the patient that relate to blood pressure, and
wherein the processor is in the sensor unit and is programmed to use the monitored signals from the patient, the pressure signal from the inflation system, and the size information from the blood pressure cuff to determine the patient's blood pressure.

3. The system of claim 1, wherein the monitor component includes a wireless transceiver and the inflation system includes a wireless transceiver and wherein the monitor component is configured to send the received size information via the monitor component's wireless transceiver to the inflation unit's wireless transceiver.

4. The system of claim 1, further comprising a sensor unit to be worn on the patient's body to monitor signals from the patient that relate to blood pressure, and wherein the processor is programmed to use the monitored signals from the patient, the pressure signal from the inflation system, and the size information from the blood pressure cuff to determine the patient's blood pressure.

5. The system of claim 1, wherein the sensor unit includes a wireless transceiver and the monitor component includes a wireless transceiver, wherein the processor is within the sensor unit, and wherein the monitor component is configured to send the received size information via the monitor component's wireless transceiver to the sensor unit's wireless transceiver.

6. The system of claim 1, wherein monitor component includes a display device and the interface in the monitor component is a graphical user interface displayed in the display device and through which the user enters the size information from the blood pressure cuff.

7. The system of claim 1, wherein the interface in the monitor component is a bar code reader and wherein the sizing indicator comprises a bar code.

8. The system of claim 1, wherein the sizing indicator is a barcode label.

9. The system of claim 8, wherein the interface in the monitor component comprises a barcode scanner.

10. The system of claim 1, wherein the inflation system includes a pump, wherein the processor is within the inflation system, and the processor is programmed to control the rate at which the pump inflates the blood pressure cuff based on the received size information.

11. The system of claim 10, wherein the processor is programmed is programmed to control the rate at which the pump inflates the blood pressure cuff based on the received size information and the information derived from the pressure signal.

12. The system of claim 1, wherein the blood pressure cuff comprises a flexible strap that includes the size indicator and the size indicator presents information about a circumference of the patient's arm about which the blood pressure cuff is placed when in use.

13. The system of claim 12, wherein the size indicator comprises a plurality of labels, each one indicating a different arm circumference, and a marker that identifies which label among the plurality of labels identifies the circumference of the patient's arm about which the blood pressure cuff is placed when in use.

14. The system of claim 1, wherein the size information is a circumference of the patient's arm around which the blood pressure cuff is placed when in use and wherein the processor is further programmed to determine a blood pressure offset value as part of determining the patient's blood pressure.

15. The system of claim 1, wherein the sizing indicator comprises an alphanumeric code encoding the size information for the blood pressure cuff.

16. The system of claim 15, wherein the monitor component includes a reader for reading the alphanumeric code of the sizing indicator.

17. The system of claim 16, wherein the monitor component includes a reader for wirelessly reading the alphanumeric code of the sizing indicator.

18. The system of claim 17, wherein the sizing indicator comprises an RFID chip, and the interface in the monitor component comprises an RFID reader.

19. A system for measuring blood pressure of a patient, said system comprising:
a blood pressure cuff including a sizing indicator that presents size information about at least one of a size of the blood pressure cuff and a size of a patient's arm about which the blood pressure cuff is placed when in use;
a monitor component including a first processor and a display device, wherein the first processor is programmed to display a graphical user interface on the display device and through which the size information from the sizing indicator is entered by a user;
an inflation system that inflates the blood pressure cuff, said inflation system also including a pressure sensor for generating a pressure signal which is a measure of the pressure applied the patient's arm by the inflation system; and
a processor programmed to use the pressure signal from the inflation system and the size information from the blood pressure cuff to determine the patient's blood pressure.

20. A system for measuring blood pressure of a patient, said system comprising:
a blood pressure cuff including a sizing indicator that presents size information about at least one of a size of the blood pressure cuff and a size of a patient's arm about which the blood pressure cuff is placed when in use;
a monitor component including sensing system for reading the size information from the sizing indicator and also including a wireless transmitter for transmitting the size information; and
an inflation system that inflates the blood pressure cuff, said inflation system including a pressure sensor, a wireless receiver, and a processor, said pressure sensor for generating a pressure signal which is a measure of the pressure applied the patient's arm by the inflation system, said wireless receiver for receiving the size information transmitted by the transmitter in the monitor component, and said processor programmed to use the pressure signal from the inflation system and the size information from the blood pressure cuff to determine the patient's blood pressure.
